(19) United States
(12) Patent Application Publication
(10) Pub. No.: US 2008/0064963 A1
(43) Pub. Date: Mar. 13, 2008

(54) Device For Detecting The Gravity Of An Illness

(76) Inventors: Matthias Schwaibold, Karlsruhe (DE); Bernhard Scholler, Karlsruhe (DE); Ludger Grote, Gothenburg (DE)

Correspondence Address: FRIEDRICH KUEFFNER, 317 MADISON AVENUE, SUITE 910, NEW YORK, NY, 10017, US

(21) Appl. No.: 11/596,616

(22) PCT Filed: Apr. 27, 2005
(86) PCT No.: PCT/DE05/00768

§ 371 Date: Aug. 21, 2007

(30) Foreign Application Priority Data
May 22, 2004 (DE) 10 2004 025 200.9

(51) Int. Cl. A61B 5/02

(52) U.S. Cl. 600/481

(57) ABSTRACT

The device is used to detect the gravity of an illness of a living being and the method is used to control the corresponding detection device. At least one sensor (1) for non-invasive measurement of at least one signal dependent upon the autonomous regulation of the cardiovascular system of the living being is connected to an evaluation device (2). The evaluation device (2) is provided with a calculation unit (4) for converting the signal into a state parameter. The evaluation unit also has an index determining device (6) for determining at least one cumulative system parameter. A comparison device (8) that analyses the index while considering other parameters of the living being is connected to an output device (9) for generating the detected gravity of the illness.

The invention concerns a device for determining the severity of an illness.

The invention also concerns a method for controlling a detection device for evaluating the severity of an illness.

In many illnesses, including, for example, illnesses related to a patient's sleep, there are dependent relationships with cardiovascular diseases. Furthermore, there are also diverse interactions accompanied by impairment of the quality of life of the patient, and causes and effects mutually affect each other.

In the past, it has been possible to determine the extent and the intensity of such interactions only statistically for a large number of patients but not for a specific individual patient.

Various measuring methods are already known for determining individual parameters related to the autonomic regulation of the cardiovascular system. Measuring methods of this type are described, for example, in U.S. Pat. No. 5,862,805, WO 91/11956, US 2002-0029000, WO 02/067776, and EP 0 995 592. However, so far no methods or devices have been disclosed which relate to a comprehensive evaluation of the measured values for comprehensive consideration of the individual factors in the determination of the severity of the illness.

In particular, there are no devices at all which make it possible to use the combination of parameters they measure to make predictions, on the basis of the existing illness (which is possibly but not necessarily diagnosed with the same device), about the individual risk of developing secondary conditions that impair the quality of life or lower life expectancy.

There are also no devices at all which make it possible to use the combination of parameters they measure to make individually optimum selections of therapeutic treatments and therapeutic dosages from the existing therapeutic treatments and dosages for the illness, which is possibly but not necessarily diagnosed with the same device.

There are also no devices at all which make it possible to use the combination of parameters they measure to make predictions about the therapeutic success with an illness, which is possibly but not necessarily diagnosed with the same device.

Therefore, the objective of the present invention is to design a device of the aforementioned type in such a way that it can be individually applied to a patient.

In accordance with the invention, this objective is achieved in such a way that at least one sensor for the noninvasive measurement of at least one signal that is dependent on the autonomic regulation of the cardiovascular system of a person is connected to an evaluation device, that the evaluation device has a calculation unit for converting the signal to a state parameter as well as an index-determining device for determining at least one cumulative system parameter, and that a comparison device that analyzes the index by considering other parameters of the person is connected to an output device for the determined severity of the illness.

In contrast to the diagnosis of an illness, the term “severity” as used in the context of the invention does not mean merely the pure frequency and intensity of the occurrence of certain pathophysiological events. Rather, the detected autonomic regulation of the cardiovascular system is used to recognize the extent of the individual risk of the tested person to suffer from secondary conditions that impair the quality of life or lower life expectancy. The determined severity of the illness can also be used to determine the optimum form of therapy and therapeutic dosage for the individual person. In addition, the success of an already existing therapy can be measured.

A further objective of the invention is to specify a method for automatically controlling a detection device in such a way that the necessary determinations and evaluations of the measured values can be carried out automatically.

In accordance with the invention, this objective is achieved in such a way that at least one signal that is dependent on the autonomic regulation of the cardiovascular system of a person is detected by a noninvasive measurement and analyzed in an evaluation device, that the evaluation unit converts the signal to a state parameter and determines an index, which is assigned to a cumulative system parameter, that the index is compared with other parameters of the person, by means of which the severity of the illness is determined, and that the determined characteristic value for characterizing the severity of the illness is output by the evaluation device.

The use of the device of the invention and the realization of the control method of the invention make it possible to acquire information about a large number of individual states of the patient by evaluation of the autonomic regulation of the cardiovascular system. These are, for example, sleep and sleep fragmentation, respiration, chemical regulation, blood pressure regulation, sympathetic and parasympathetic activities, and the reactivity of the cardiovascular system.

By evaluating the associated parameters, it is possible to determine the interactions of a large number of pathophysiological processes. In addition, it is possible that, with the use of a significantly reduced number of measuring sensors compared to the prior art, information can be obtained that is comparable to that obtained with methods that are already well known, for example, with respect to respiratory disturbances.

Signal evaluation over a predetermined period of time is assisted by connecting the sensor with a first memory unit for storing a detected measuring signal.

Signal processing is also assisted by connecting the calculation unit with a second memory unit for storing the determined state parameter.

To carry out an analysis of variance, it is proposed that the index-determining device have a variance analyzer for analyzing the variance of the state parameter with respect to time.

To further improve the evaluation possibilities, it is proposed that the index-determining device be provided with a third memory unit for storing the calculated index.

In a typical evaluation sequence, the amplitude variation of the measuring signal is evaluated.

It is also proposed that a slope of the measuring signal be evaluated.

In another variant of the invention, a frequency of the measuring signal is evaluated.

With respect to predictive sensitivity, it was found to be especially advantageous to evaluate an intensity of variation of the measuring signal.

A comprehensive signal evaluation can be effected by carrying out a pattern recognition.

One possibility for signal acquisition is the evaluation of an EEG signal.

The evaluation of an ECG signal is likewise proposed.

Another measurement variant consists in the evaluation of the oxygen saturation of the blood.

To acquire respiratory parameters, it is provided that the respiratory pattern be evaluated.

A measuring principle that is easily realized is the evaluation of the optical density of at least one body region.

In accordance with a typical evaluation method, it is provided that a signal evaluation be carried out with respect to existing periodic signal components.

Especially informative signal patterns can be evaluated by performing an analysis with respect to a maximum signal change.

Especially high predictive quality is achieved if the evaluation unit determines an autonomic resting profile of the regulation of the cardiovascular system. In this regard, the autonomic resting profile is connected with at least one of the following parameters: depth of sleep, sleep fragmentation, activity of the parasympathetic nervous system absolutely or in relation to the sympathetic nervous system, blood pressure, and vascular compliance.

In particular, it was found to be advantageous that the index is assigned to a cumulative autonomic resting intensity of the regulation of the cardiovascular system.

With respect to the determination of respiratory activities, it is proposed that, in the determination of the autonomic resting profile, a respiratory activity be taken into consideration, or the amplitude of the respiration-correlated oscillations be evaluated in the detected parameter.

The detection of especially informative events can be achieved by carrying out a signal analysis with respect to a period of activation of the autonomic system.

A further increase in the predictive quality can be realized if, in the evaluation of periods of activation of the autonomic system, at least one additional respiratory parameter is evaluated. The increase in predictive quality can be realized by comparing the change, with respect to intensity, type, and time sequence, in various detected parameters or in various parameters derived from the detected parameters at the time of an activation of the autonomic system.

To eliminate disturbances or singular events, it is proposed that, in the determination of the index, the cumulative number and the intensity of the activation periods of the autonomic system be taken into consideration.

In a simple measurement setup, the sensor detects the heart rate.

In particular, it is proposed that the sensor detect the variability of the heart rate.

Furthermore, it is also possible for the sensor to detect the pulse transit time.

Alternatively, it is possible for the sensor to detect the pulse amplitude.

To help eliminate disturbances, it is useful to evaluate a mean value of the measuring signal detected by the sensor, which mean value is determined over a predetermined period of time. The elimination of disturbances can be further improved by performing an artifact detection and elimination before the evaluation of the detected parameters.

Detection of short-term signal variations is assisted by evaluating a maximum value of the measuring signal detected by the sensor. This maximum value is the maximum value that occurs over a predetermined interval of time.

A measuring method that is simple to use consists in carrying out the signal acquisition with the use of photoplethysmography.

An increase in system sensitivity can be achieved by carrying out the signal evaluation within at least one predetermined frequency band.

To allow interactions to be taken into consideration, it is provided that at least one additional physical parameter is evaluated by the evaluation unit.

It is proposed that, for example, the age of the person be evaluated as an additional physical parameter.

It is also possible to evaluate the sex of the person as an additional physical parameter.

Alternatively or additionally, it is also possible to evaluate the weight of the person as an additional physical parameter. Alternatively or additionally, it is also possible to evaluate one or more already well-known risk factors for cardiovascular disease as additional physical parameters.

Alternatively or additionally, it is also possible to evaluate one or more already well-known factors that affect autonomic regulation, especially medication, as additional physical parameters.

Alternatively or additionally, it is also possible to evaluate one or more additionally detected parameters, e.g., arterial oxygen saturation, as additional physical parameters.

Temporally anterior events can be taken into consideration by evaluating the case history of the person as an additional physical parameter.

A further increase in predictive quality can be achieved by evaluating the medication of the person as an additional physical parameter.

Specific embodiments of the invention are schematically illustrated in the accompanying figures.

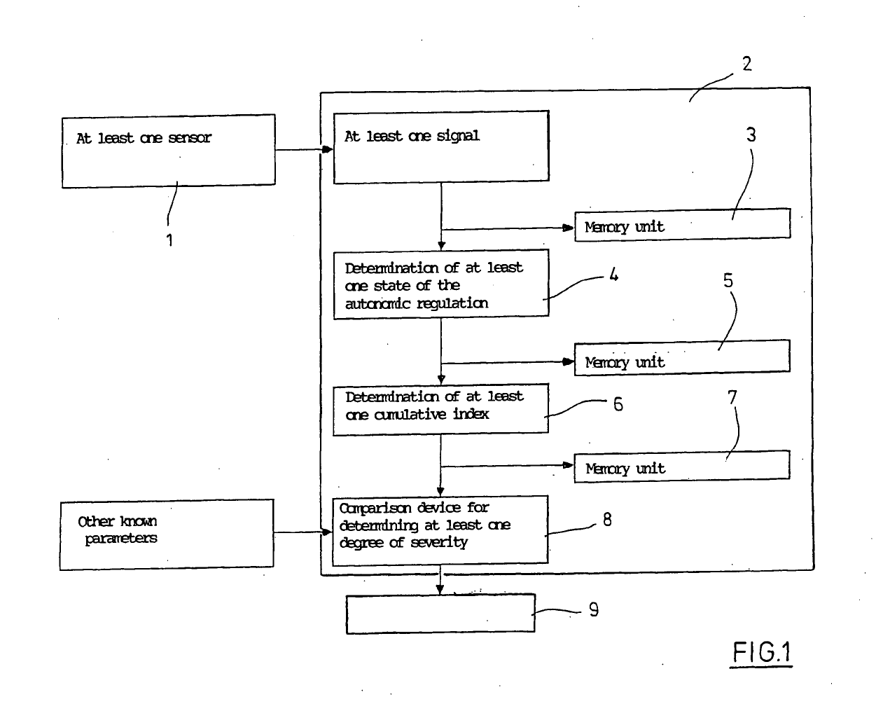

FIG. 1 shows a functional block diagram illustrating a typical equipment setup.

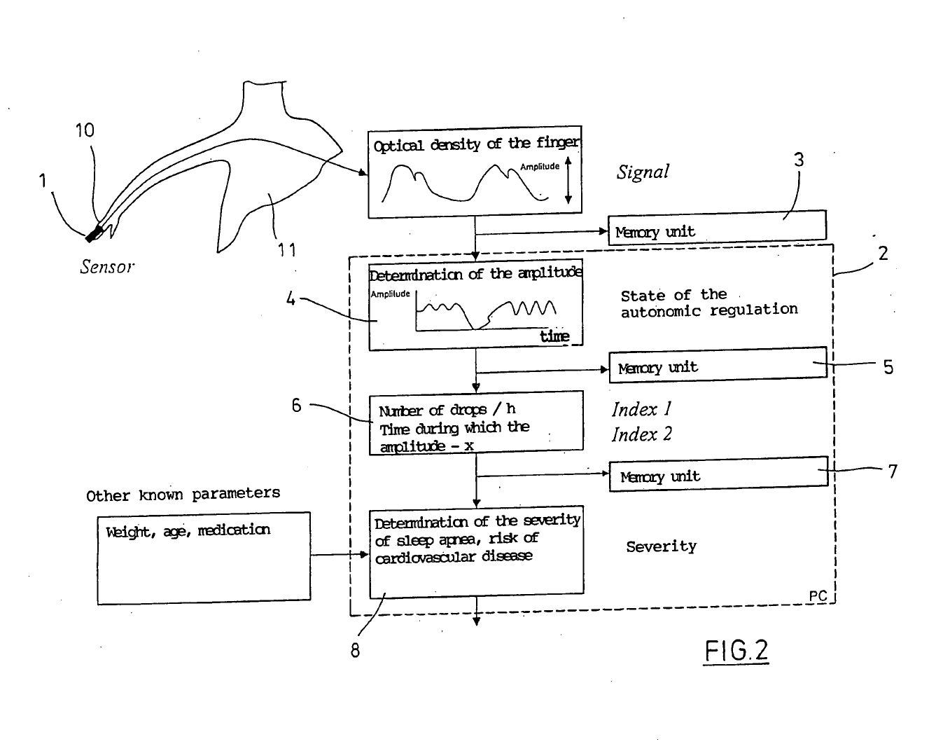

FIG. 2 shows a flow chart illustrating the performance of typical process steps in the processing of the measured values.

FIG. 1 shows a specific embodiment of the structural design of the device for determining the severity of an illness. For the sake of simplicity, an embodiment is shown in which only one sensor (1) is used. It is connected to an evaluation device (2).

The signal detected by the sensor (1) is first stored in a first memory unit (3) for at least the duration of a subsequent signal processing step. The evaluation unit (2) is connected to a calculation unit (4), which converts the signal detected by the sensor (1) to a state parameter of the autonomic regulation of the cardiovascular system. The state parameter is stored in a second memory unit (5).

The calculation unit (4) is connected to an index-determining device (6), which determines a cumulative system parameter of the autonomic regulation of the cardiovascular system. The index that has been determined is stored in a third memory unit (7). The index thus determined is analyzed by a comparison device (8), which takes into consideration at least one other parameter of the person to be tested, and is transmitted to an output device (9).

FIG. 2 shows a specific embodiment in which a sensor (1) for the optical detection of the density of a finger (10) is placed in the vicinity of the finger (10) of a patient (11). The amplitude of the variation of density as a function of time is determined. The optical density of the finger (10) typically varies as a function of the blood circulation caused by the pulse of the patient (11). In addition, the strength of the pulsation and the mean optical density vary with the respiration of the patient and upon activation of the sympathetic and parasympathetic nervous systems.

In this embodiment, the first memory unit (3) is arranged outside the evaluation unit (2), while the second memory unit (5) and the third memory unit (7) are realized as part of the evaluation unit (2). The calculation unit (4) determines the variation of the amplitude of an associated state parameter of the autonomic regulation of the cardiovascular system. The index-determining device (6) determines the number of amplitude drops per hour and the time during which the given amplitude values are above a predetermined limit. Accordingly, in this particular embodiment, a first index and a second index are determined.

Taking into account the first index and the second index and taking into account other known parameters of the patient (11), for example, weight, age, or medication, the comparison device (8) determines the severity of an existing illness or a disease risk. In the present embodiment, the severity of sleep apnea is determined.

In particular, the variability of the determined parameters during normal sleep phases and sleep phases that are fragmented by respiratory events provides information about impairment of autonomic regulation that already exists or is to be expected and about the risk of secondary conditions. Both the intensity of the cyclic variability, which is correlated with, among other things, breathing, and the intensity of the transient variability, which is associated with, among other things, reactions that occur upon waking, are evaluated and compared with the individual variability to be expected on the basis of age, weight, case history, etc.

In addition, this makes it possible to determine the individual need for therapy and the most suitable form of therapy and therapeutic dosage: medication, CPAP therapy, CPAP therapy with different inspiratory and expiratory pressure levels, and CPAP therapy with automatic pressure adaptation with or without obstruction detection.

In this regard, it is not necessary for the illness to be diagnosed with the same device. This can be done, e.g., by conventional measurement of respiration and respiratory gas exertion. However, by including the parameter of oxygen saturation, a special design of the same device can also be used to determine an AHI with differentiation of central and obstructive events. In this regard, phases with insufficient respiration are recorded on the basis of the saturation. In the case of an unclear course, these phases can be confirmed by a sympathetically related drop in blood circulation or an increase in heart rate during or at the end of the event. If the amplitude of the respiration-related variations of the measured parameter increases during the event, then the event is an obstructive event. If it decreases, then the event is a central event.

Automated evaluation of the severity of various conditions can be performed with the use of the device explained above and the method for controlling the device. Examples of such conditions are respiratory disturbances during sleep, such as obstructive sleep apnea, central sleep apnea, periodic respiration, Cheyne-Stokes respiration, snoring, or flow limitation. Moreover, it is possible to use the device and method of the invention in connection with cardiovascular diseases, such as hypertension, chronic cardiac insufficiency, coronary heart disease, stroke, myocardial infarction, arrhythmia, cardiomyopathy, or sudden cardiac death. They can also be used in connection with general respiratory tract diseases, such as COPD or asthma. Another area of application is neuropathy.

It is possible to perform an analysis either with respect to the existence or the nonexistence of a certain illness, according to the particular setting of the apparatus. It is also possible to determine the risk of another disease, depending on the severity of a first disease.

1. A device for determining the severity of an illness, wherein at least one sensor for the noninvasive measurement of at least one signal that is dependent on the autonomic regulation of the cardiovascular system of a person is connected to an evaluation device, where the evaluation device has a calculation unit for converting the signal to a state parameter as well as an index-determining device for determining at least one cumulative system parameter, and where a comparison device that analyzes the index by considering other parameters of the person is connected to an output device for the determined severity of the illness.

2. A device in accordance with claim 1, wherein the sensor (1) is connected with a first memory unit (3) for storing a detected measuring signal.

3. A device in accordance with claim 1, wherein the calculation unit (4) is connected with a second memory unit (5) for storing the determined state parameter.

4. A device in accordance with 3, claim 1, wherein the index-determining device (6) has a variance analyzer for analyzing the variance of the state parameter with respect to time.

5. A device in accordance with claim 1, wherein the index-determining device (6) is provided with a third memory unit (7) for storing the calculated index.

6. A method for controlling a detection device for evaluating the severity of an illness, wherein at least one signal that is dependent on the autonomic regulation of the cardiovascular system of a person is detected by a noninvasive measurement and analyzed in an evaluation device, where the evaluation unit converts the signal to a state parameter and determines an index, which is assigned to a cumulative system parameter, where the index is compared with other parameters of the person, by means of which the severity of the illness is determined, and where the determined characteristic value for characterizing the severity of the illness is output by the evaluation device.

7. A method in accordance with claim 6, wherein the amplitude variation of the measuring signal is evaluated.

8. A method in accordance with claim 6, wherein a slope of the measuring signal is evaluated.

9. A method in accordance with claim 6, wherein a frequency of the measuring signal is evaluated.

10. A method in accordance with claim 6, wherein an intensity of variation of the measuring signal is evaluated.

11. A method in accordance with claim 6, wherein a pattern recognition is carried out.

12. A method in accordance with claim 6, wherein an EEG signal is evaluated.

13. Amended) A method in accordance with claim 6, wherein an ECG signal is evaluated.

14. A method in accordance with claim 6, wherein the oxygen saturation of the blood is evaluated.

15. A method in accordance with claim 6, wherein the respiratory pattern is evaluated.

16. A method in accordance with claim 6, wherein the optical density of at least one body region is evaluated.

17. A method in accordance with claim 6, wherein a signal evaluation is carried out with respect to existing periodic signal components.

18. A method in accordance with claim 6, wherein an analysis with respect to a maximum signal change is performed.

19. A method in accordance with claim 1, wherein an autonomic resting profile of the regulation of the cardiovascular system is determined by the evaluation unit (2).

20. A method in accordance with claim 6, wherein the index is assigned to a cumulative autonomic resting intensity of the regulation of the cardiovascular system.

21. A method in accordance with claim 6, wherein a respiratory activity is taken into consideration in the determination of the autonomic resting profile.

22. A method in accordance with claim 6, wherein a signal analysis is carried out with respect to a period of activation of the autonomic system.

23. A method in accordance with claim 6, wherein in the evaluation of periods of activation of the autonomic system, at least one additional respiratory parameter is evaluated.

24. A method in accordance with claim 6, wherein in the determination of the index, the cumulative number and the intensity of the activation periods of the autonomic system are taken into consideration.

25. A method in accordance with claim 6, wherein the sensor (1) detects the heart rate.

26. A method in accordance with claim 6, wherein the sensor (1) detects the variability of the heart rate.

27. A method in accordance with claim 6, wherein the sensor (1) detects the pulse transit time.

28. A method in accordance with claim 6, wherein the sensor (1) detects the pulse amplitude.

29. A method in accordance with claim 6, wherein a mean value of the measuring signal detected by the sensor (1) is evaluated, which mean value is determined over a predetermined period of time.

30. A method in accordance with claim 6, wherein a maximum value of the measuring signal detected by the sensor (1) is evaluated, which maximum value is the maximum value that occurs over a predetermined interval of time.

31. A method in accordance with claim 6, wherein the signal acquisition is carried out with the use of photoplethysmography.

32. A method in accordance with claim 6, wherein the signal evaluation is carried out within at least one predetermined frequency band.

33. A method in accordance with claim 6, wherein at least one additional physical parameter is evaluated by the evaluation unit (2).

34. A method in accordance with claim 33, wherein the age of the person is evaluated as an additional physical parameter.

35. A method in accordance with claim 33, wherein the sex of the person is evaluated as an additional physical parameter.

36. A method in accordance with claim 33, wherein the weight of the person is evaluated as an additional physical parameter.

37. A method in accordance with claim 33, wherein the case history of the person is evaluated as an additional physical parameter.

38. A method in accordance with claim 33, wherein the medication of the person is evaluated as an additional physical parameter.
